(19) United States
(12) Patent Application Publication
(10) Pub. No.: US 2002/0192170 A1
(43) Pub. Date: Dec. 19, 2002

(54) Liquid for lip care and lip coloring

(76) Inventors: Tatiana Appel, Oberasbach (DE); Gerhard Lugert, Nurnberg (DE); Wolfgang Schwarz, Ansbach (DE)

Correspondence Address: LERNER A. GREENBERG, Post Office Box 2480, Hollywood, FL, 33022-2480, US

(21) Appl. No.: 10/115,844

(22) Filed: Apr. 3, 2002

(30) Foreign Application Priority Data
Apr. 3, 2001 (DE) 101 16 497.1-41

(51) Int. Cl. A61K 7/025

(52) U.S. Cl. 424/64

(57) ABSTRACT

A liquid for lip care and lip coloring contains water and a moisture retainer as main ingredients. A polyoxyethylene glycerin fatty acid ester is also provided.

### BACKGROUND OF THE INVENTION

Field of the Invention

The invention relates to a liquid for lip care and lip coloring. Conventional lipsticks comprise a cartridge of oily or waxy consistency. U.S. Pat. No. 5,756,014 to Mathur describes a lipstick containing polyoxyethylene glycerin monostearate or polyoxyethylene glycerin monooleate. A relatively large amount must be applied to the skin in order to achieve sufficient coloring. A disadvantage of this is that the application easily smears and comes off. Furthermore, it is not easy to achieve a precisely linear application with the relatively thick lipstick cartridges. This can be managed more easily with capillary applicators utilizing liquid formulas. A compound for lip coloring described in U.S. Pat. No. 2,548,970 to Grate contains benzoic resin, hexadecanol, and lecithin, in addition to a volatile solvent such as ethanol or isopropyl alcohol. Formulas with film formers are described in European Patent No. 853 940 B1 and U.S. Pat. No. 4,795,631 to Sheehan. German Published, Non-Prosecuted Patent Application DE 196 279 31 A1 describes a lip colorant with a mixture of water and isopropyl alcohol as the solvent.

### SUMMARY OF THE INVENTION

It is accordingly an object of the invention to provide a liquid for lip care and lip coloring that overcomes the hereinafore-mentioned disadvantages of the heretofore-known devices of this general type and that has an alternative composition that can be applied using a capillary applicator.

With the foregoing and other objects in view, there is provided, in accordance with the invention, a liquid for lip care and lip coloring. The liquid includes water, a moisture retainer, and a polyoxyethylene glycerin fatty acid ester.

In accordance with a further object of the invention, the water and said moisture retainer are main ingredients.

In accordance with a further object of the invention, the liquid includes polyoxyethylene glycerin stearin acid ester.

In accordance with a further object of the invention, the liquid has, by weight %, 2 to 40% of the moisture retainer, 0.1 to 10% of the polyoxyethylene glycerin fatty acid ester, 0 to 22% additives, and ad 100% of the water.

In accordance with a further object of the invention, the liquid includes polyether-modified polysiloxane.

In accordance with a further object of the invention, the liquid has, by weight %, 2 to 40% of the moisture retainer, 0.1 to 10% of the polyoxyethylene glycerin fatty acid ester, 0.2 to 3% of the polyether-modified polysiloxane, 0 to 22% of additives, and ad 100% of the water.

In accordance with a further object of the invention, the liquid has, by weight %, 0 to 5% of a flavoring, 0 to 5% of a main skin-care agent, 0 to 10% of dyes, and 0 to 2% of a preservative.

In accordance with a further object of the invention, the liquid has, by weight %, 10.0 to 35.0% of the moisture retainer, 4 to 10% of the polyoxyethylene glycerin fatty acid ester, 0 to 1.0% of the main skin-care agent, 0 to 5.0% of the dyes, 0 to 4.0% of the flavoring, 0 to 1.0% of the preservative, and ad 100% water.

In accordance with a further object of the invention, the liquid has, by weight %, 10.0 to 35.0% of the moisture retainer, 4 to 10% of the polyoxyethylene glycerin fatty acid ester, 0.5 to 3.0% of the polyether-modified polysiloxane, 0 to 1.0% of the main skin-care agent, 0 to 5.0% of the dye; 0 to 4.0% of the flavoring, 0 to 1.0% of the preservative, and ad 100% of water.

In accordance with a further object of the invention, the main skin-care ingredient can be aloe vera agent, tocopherol, and/or shea butter.

In accordance with a further object of the invention, the liquid has a viscosity no greater than 20 mPa·s.

In accordance with a further object of the invention, the moisture retainer can be an alcohol, a glycerin, and/or a glycol.

This object is inventively achieved by a liquid containing water and at least one moisture retainer as the main ingredients, as well as at least one polyoxethylene glycerin fatty acid ester. Possible moisture retainers include alcohols, glycerin, or glycols. It has been demonstrated that a combination of these substances produces a liquid that dries quickly and thus soaks into the skin without drying the skin. The liquid can be applied to the skin without any problem using a capillary tip such as a fiber tip of an applicator. A polyoxyethylene glycerin stearin acid ester is preferably used. In a preferred exemplifying embodiment, the liquid contains 2-40% moisture retainer, 0.1-10% polyoxyethylene glycerin fatty acid ester, and up to 22% additives (such as skin-care additives) by weight, the remainder being water.

An improvement with respect to the permanence and gloss of the liquid and its compatibility with lipophilic components is achieved by the addition of polyether-modified polysiloxane. In a formulation with the above content groups, the proportion of polysiloxane is 0,2 to 3% by weight. Particularly preferred compounds contain 10 to 30% moisture retainer, 4 to 10% polyoxyethylene glycerin fatty acid ester, and potentially 0.5 to 3% polyether-modified polysiloxane by weight. For all compounds, a maximum viscosity of 20 mPa·s (Brookfield, T=20° C.) is set. Such a liquid can be easily inserted into capillary applicators, i.e. applicators that work according to the same principle as fiber recorders. In all formulations, the dyes are preferably dyes from the “acid dye” group that may be used in cosmetics.

Other features that are considered as characteristic for the invention are set forth in the appended claims.

Although the invention is illustrated and described herein as embodied in a liquid for lip care and lip coloring, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the following examples.

Description of the Preferred Embodiments:

### EXAMPLE 1

### Colorless Skin-Care Liquid

| | | |
|---|---|---|
| Methyl-4-hydroxybenzoate | 0.20% | by weight |
| Propylene glycol | 15.00% | |
| Polyoxyethylene glycerin | 8.00% | |
| stearin acid ester | | |
| (CAS 68553-11-7) | | |
| Water | 74.48% | |
| Polyether-modified polysiloxane | 1.50% | |
| Aloe Vera (powder) | 0.70% | |
| Tocopherol | 0.10% | |
| Citric acid | 0.02% | |

In the manufacturing process, the above components are mixed together, for instance in the above order. Methyl-4-hydroxybenzoate serves as a preservative. The skin-care additives are tocopherol, citric acid as a synergist (for tocopherol), and aloe vera. The polyoxyethylene glycerin stearin acid ester, which can be abbreviated as PEG-30-glyceriyl stearate, can be acquired from the company Goldschmidt under the trademark TAGATO S. The polyether-modified polysiloxane can be acquired from the same manufacturer under the trademark ABIL® B 8851.

### EXAMPLE 2

### Violet Lip Coloring Liquid with Flavoring

| | | |
|---|---|---|
| Methyl-4-hydroxybenzoate | 0.50% | by weight |
| Ethanol | 20.00% | |
| Polyoxyethylene glycerin | 6.00% | |
| stearin acid ester | | |
| (CAS 68553-11-7) | | |
| Glycerin | 10.00% | |
| Water | 69.62% | |
| Polyether-modified polysiloxane | 1.00% | |
| Rum flavoring | 2.00% | |
| Red dye (CT 45410) | 0.50% | |
| Orange dye (CI 15985) | 0.32% | |
| Blue dye (CI 42090) | 0.06% | |

### EXAMPLE 3

### Brown Lip Coloring Liquid

| | | |
|---|---|---|
| Methyl-4-hydroxybenzoate | 0.50% | by weight |
| Ethanol | 20.00% | |
| Polyoxyethylene glycerin | 6.00% | |
| stearin acid ester | | |
| (CAS 68553-11-7) | | |
| Glycerin | 10.00% | |
| Water | 59.40% | |
| Polyether-modified polysiloxane | 1.00% | |
| Aloe Vera (powder) | 0.50% | |
| Red dye (CI 45380) | 1.14% | |
| Orange dye (CI 15985) | 1.26% | |
| Blue dye (CI 42090) | 0.20% | |

### EXAMPLE 4

### Red Lip Coloring Liquid

| | | |
|---|---|---|
| Methyl-4-hydroxybenzoate | 0.50% | by weight |
| Ethanol | 20.00% | |
| Polyoxyethylene glycerin | 5.50% | |
| stearin acid ester | | |
| (CAS 68553-11-7) | | |
| Glycerin | 10.00% | |
| Water | 61.98% | |
| Red dye (CI 45380) | 0.90% | |
| Orange dye (CI 15985) | 1.12% | |

We claim:

1. A liquid for lip care and lip coloring, comprising:
water,
a moisture retainer, and
a polyoxyethylene glycerin fatty acid ester.

2. The liquid according to claim 1, wherein said water and said moisture retainer are main ingredients.

3. The liquid according to claim 1, including polyoxyethylene glycerin stearin acid ester.

4. The liquid according to claim 1, having by weight %:
2 to 40% of said moisture retainer;
0.1 to 10% of said polyoxyethylene glycerin fatty acid ester;
0 to 22% additives; and
ad 100% of said water.

5. The liquid according to claim 1, including polyether-modified polysiloxane.

6. The liquid according to claim 5, having by weight %:
2 to 40% of said moisture retainer;
1 to 10% of said polyoxyethylene glycerin fatty acid ester;
2 to 3% of said polyether-modified polysiloxane;
0 to 22% of additives; and
ad 100% of said water.

7. The liquid according to claim 4, having by weight %:
0 to 5% of a flavoring;
0 to 5% of a main skin-care agent;
0 to 10% of dyes; and
0 to 2% of a preservative.

8. The liquid as claimed in claim 7, having by weight %:
10.0 to 35.0% of said moisture retainer;
4 to 10% of said polyoxyethylene glycerin fatty acid ester;
0 to 1.0% of said main skin-care agent;
0 to 5.0% of said dyes;
0 to 4.0% of said flavoring;
0 to 1.0% of said preservative; and
ad 100% water.

9. The liquid according to claim 7, having by weight %:
10.0 to 35.0% of said moisture retainer;
4 to 10% of said polyoxyethylene glycerin fatty acid ester;
0.5 to 3.0% of said polyether-modified polysiloxane;
0 to 1.0% of said main skin-care agent;
0 to 5.0% of said dye;
0 to 4.0% of said flavoring;
0 to 1.0% of said preservative; and
ad 100% of water.

10. The liquid according to claim 7, wherein said main skin-care ingredient is selected from the group consisting of aloe vera agent, tocopherol, and shea butter.

11. The liquid according to claim 1, having a viscosity no eater than 20 mpa·s.

12. The liquid according to claim 1, wherein said moisture retainer is selected from the group consisting of an alcohol, a glycerin, and a glycol.
